(19) United States
(12) Patent Application Publication
(10) Pub. No.: US 2023/0339839 A1
(43) Pub. Date: Oct. 26, 2023

(54) SYNTHESIS METHOD OF ALPHA-HYDROXYCARBOXYLIC ACID ESTER

(71) Applicant: Guangxi Forestry Research Institute, Nanning (CN)

(72) Inventors: Zhonglei Meng, Nanning (CN); Rongxiu Qin, Nanning (CN); Shouna Gao, Qionghai (CN); Guojiao Hu, Nanning (CN); Rusi Wen, Nanning (CN); Zhongqiu Liao, Nanning (CN); Zhongyun Liang, Nanning (CN); Lihong Hu, Nanjing (CN); Yonghong Zhou, Nanjing (CN)

(21) Appl. No.: 18/094,155

(22) Filed: Jan. 6, 2023

Related U.S. Application Data
(63) Continuation of application No. PCT/CN2022/133407, filed on Nov. 22, 2022.

(30) Foreign Application Priority Data
Apr. 24, 2022 (CN) 2022104345782

(51) Int. Cl. C07C 67/08; C07C 67/54; C07C 67/48; B01J 37/00; B01J 21/02; B01D 17/04; B01D 3/10

(52) U.S. Cl. CPC C07C 67/08; C07C 67/54; C07C 67/48; B01J 37/009; B01J 21/02; B01D 17/042; B01D 3/10

(57) ABSTRACT

A synthesis method of alpha-hydroxycarboxylic acid ester is provided. In the method, an alpha-hydroxycarboxylic acid and an alcohol are taken as raw materials, one or more of boric acid, metaboric acid, pyroboric acid, and boric anhydride is taken as a catalyst, and one of cyclohexane, toluene, benzene, and alcohols is taken as a water-carrying agent. A reaction temperature is kept in range of 100-160° C., produced water is separated by an oil-water separator, and the alpha-hydroxycarboxylic acid ester is synthesized directly. After an esterification reaction, the water-carrying agent and unreacted alcohol are distilled by vacuum fractionation to obtain a crude product, and refined alpha-hydroxycarboxylic acid ester is obtained by performing neutralization, water washing, and vacuum rectification on the crude product. The alpha-hydroxycarboxylic acid ester prepared by the method has high purity and light color, and can be used as green solvent, surfactant, chiral resolution agent and plasticizer.

### TECHNICAL FIELD

The disclosure relates to the technical field of fine chemicals production, in particular to a synthesis method of alpha-hydroxycarboxylic acid ester (also referred to as α-hydroxycarboxylic acid ester).

### BACKGROUND

Alpha-hydroxycarboxylic acid ester is a kind of important fine chemical products, which are widely used in daily chemical, pharmaceutical, plastic and other industries. For example, butyl lactate has special aromas of wine, fruit and milk, and it is a “green chemical product” with great development value and application prospect due to its non-toxic, good water solubility, low volatility, low irritation and biodegradability. Ethyl-(S)-(+)-mandelate is an important chiral drug intermediate which can be widely used in the synthesis of chiral acids, chiral alcohols, chiral amines, chiral amino alcohols, chiral mercaptans, and others. N-butyl L-mandelate or n-butyl D-mandelate can be used for chiral separation. Dibutyl tartrate can be used as an extractant of chiral separation, and as a plasticizer of cellulose nitrate and cellulose acetate. Especially when the dibutyl tartrate is mixed with tritolyl phosphate in the cellulose nitrate or with benzyl alcohol in the cellulose acetate, a stable water-resistant film can be generated. Citrates, such as tributyl citrate (TBC), acetyl tributyl citrate (ATBC), trioctyl citrate (TOC), acetyl trioctyl citrate (ATOC), are a kind of important environmental plasticizers. Malate is a colorless and transparent viscous liquid with a certain odor, it is widely used as a pharmaceutical intermediate or a surfactant. Fatty alcohol malate with a short carbon chain, such as diethyl malate, is an indispensable intermediate for synthesis of some drugs including L-carnitine for weight loss products and aminopunavir, and the diethyl malate highly effective mosquito repellent. Fatty alcohol malate with a long carbon chain, such as diisostearyl malate (also referred to as bis(16-methylheptadecyl) malate), is a surfactant with good wetting effect, and can be used as the raw material of cosmetics.

Preparation methods of lactate ester are as follows.

A Chinese patent document with publication number CN102030644A discloses a preparation method of butyl lactate: adding lactic acid, butanol, toluene and a catalyst into a flask according to a certain proportion; connecting a water knockout vessel; heating and performing a reflux reaction for a certain period of time; decompressing and evaporating so as to recycle the toluene and the butanol; after evaporation, cooling a reaction system to normal temperature; and removing the catalyst by filtering so as to obtain the butyl lactate. A Chinese patent document with publication number CN103102269A discloses a preparation method of high purity butyl lactate, and the method includes: taking industrial lactic acid and n-butyl alcohol as raw materials, and adopting benzene as a water-carrying agent, subjecting them to an esterification reaction in the presence of a catalyst, then carrying out vacuum distillation and refining, thus obtaining the high purity butyl lactate; specifically, the catalyst is a composite catalyst of potassium hydrogen sulfate and ferric trichloride, or a composite catalyst of p-toluenesulfonic acid and sodium hydrogen sulfate. A Chinese patent document with publication number CN107954867A discloses a method for synthesizing high-purity butyl lactate, and relates to the technical field of organic chemical synthesis. The method includes the steps of: adopting lactic acid as a raw material, performing a reaction with the lactic acid and alkaline inorganic matter at a ratio in water to obtain lactate, distilling to remove water, performing a reaction with the lactate and n-butyl halide, and washing and performing rectification to obtain the high-purity butyl lactate. A literature document “Synthesis of n-Octyl Lactate Catalyzed with Ferric Solid Superacid” (Technology & Development of Chemical Industry, 2004, pages 1-3, Vol. 33, No. 6) discloses the most suitable reaction conditions for the synthesis of n-octyl lactate when the ferric solid superacid is taken as a catalyzer. Specifically, cyclohexane is taken as a water carrying agent, a molar ratio of lactic acid to 1-octanol is 1:1.35, a dosage of the catalyst is 0.80 grams (g) for per 0.2 moles (mol) of lactic acid, a reaction temperature is 140° C. and reaction time is 60 minutes, and an esterification yield can reach 98.6%.

Preparation methods of mandelate ester are as follows.

A Chinese patent document with publication number CN102719496A discloses a preparation method of (S)-(+)-ethyl mandelate form ethyl benzoylformate by biocatalysis taking Saccharomyces cerevisiae with CGMCC No. 2266 as a biocatalyst. A literature document “A new synthesis method of D/L-ethyl mandelate” (Fine and Specialty Chemicals, 2014, pages 42-44, Vol. 22, No. 6.) discloses that D/L-ethyl mandelate is synthesized using mandelonitrile, absolute alcohol, and concentrated sulfuric acid by means of alcoholysis. A literature document “Synthesis of n-Butyl L-Mandelate” (Chemical World, 2010, pages 691-692, Vol. 51, No. 11.) discloses that a chiral resolving reagent, n-butyl L-mandelate, is synthesized by catalysis of p-toluenesulfonic acid while L-mandelic acid and n-butanol are taken as raw materials.

Preparation methods of tartrate ester are as follows.

A Chinese patent document with publication number CN102001945A discloses a method for synthesizing ethyl tartrate with silicon chloride. A literature document “Synthesis of isobutyl L-tartrate” (Journal of Changsha University of Science and Technology (Natural Science), 2007, pages 91-93, Vol. 4, No. 4.) discloses a chiral resolving reagent, isobutyl L-tartrate, is synthesized while L-tartaric acid and isobutyl alcohol are taken as raw materials; effects such as dosage of water-carrying agent, a molar ratio of isobutyl alcohol to L-tartaric, kinds of catalyst, and dosage of catalyst are discussed; and the results show that the best catalyst is p-toluenesulfonic acid, the best dosage of the catalyst is 1 g under the molar ratio of isobutyl alcohol to L-tartaric is 3:1, and the yield of isobutyl L-tartrate is up to 99.4% under the dosage of the water-carrying agent (i.e., toluene) is 60 milliliters (mL). A literature document “Synthesis of n-butyl L-tartrate” (Chemical Engineer, 2007, pages 1-2, No. 4.) discloses a chiral resolving reagent, n-butyl L-tartrate, is synthesized while L-tartaric acid and n-butanol are taken as raw materials; the structure of the n-butyl L-tartrate is confirmed by infrared (IR); optimum reaction conditions obtained by orthogonal tests are as follows: 0.1 mol L-tartaric acid, a molar ratio of L-tartaric acid to n-butanol is 1.0:2.8, 0.5 g p-toluenesulfonic acid, 50 mL toluene, stirring at a low speed, and refluxing for about 5 hours; and the yield of n-butyl L-tartrate is over 93% and the esterification rate is up to 99%.

Preparation methods of citrate ester are as follows.

A Chinese patent document with publication number CN102336667A discloses a preparation method of triethyl citrate, and the triethyl citrate is prepared by esterification, polymerization and neutralization while raw materials are citric acid, ethanol, p-toluenesulfonic acid, phosphorous acid and tetraethyl titanate. A Chinese patent document with publication number CN101125814A discloses a method for synthesizing acetyl tributyl citrate through ionic catalysts. A Chinese patent document with publication number CN104744245A discloses an integrated synthetic process of synthesizing acetyl tributyl citrate by using an activated carbon immobilized p-toluenesulfonic acid catalyst. A Chinese patent document with publication number CN101391957A discloses a method for preparing tributyl citrate by the catalysis of rare-earth salt binary-compound solid acid, and a catalyst is filtered from the reaction mixture and can be reused. A Chinese patent document with publication number CN101735055A discloses a method for producing tirbutyl citrate, and the tirbutyl citrate is prepared by dual catalysts of sulfamic acid and tetrabutyl titanate. A Chinese patent document with publication number CN112209824A discloses a preparation method of a citrate mixture, and the citrate mixture with different ratios can be obtained by adjusting ratios of added butanol and added octanol.

Preparation methods of malate ester are as follows.

A Chinese patent document with publication number CN106187755A discloses a method for synthesizing diisostearyl malate, the method mainly uses malic acid and diisostearic alcohol to synthesize the diisostearyl malate under action of a solid catalyst which is a compound oxide composed of oxides of non-metallic element P, alkali metal element Cs, transition metal elements V. Cu and Mo and rare-earth metal elements La and Nd; a reaction temperature is in a range of 180° C.-240° C., a molar ratio of the malic acid to the diisostearic alcohol is 1:2.355, a dosage of the solid catalyst is 2%, and a water carrying agent is cyclohexane. A Chinese patent document with publication number CN101239912A discloses a method for synthesizing dibutyl malate, which uses malic acid and n-butanol as reactants, and uses naphthalene sulfonic acid methylal as a catalyst during a synthesis process of the dibutyl malate; the catalyst is filtered from reacted materials after a reaction, and the filtrate is washed with water, alkali and distilled to obtain the dibutyl malate. A Chinese patent document with publication number CN109999843A discloses a solid superacid catalyst for malic acid esterification; the catalyst contains a main body SO₄²⁻ and an oxide carrier containing Lewis acid sites, a conversion rate of the malic acid catalyzed by the catalyst reaches 100%, and yield of malate diester is over 95%. A literature document “Synthesis of diisoamyl malate by single-mode focusing microwave” (Jiangxi Chemical, 2017, pages 132-134, No. 4) discloses that an Antonio Monowave 300 single-mode sealed microwave chemical synthesis reactor is used to synthesize diisoamyl malate while malic acid and isoamyl alcohol are taken as raw materials and concentrated sulfuric acid is taken as a catalyst. A literature document “Synthesis of Dibutyl Malate with Sollid Superacid SO₄²⁻/TiO₂ as Catalyst” (Technology & Development of Chemical Industry, 2008, pages 12-14, Vol. 37, No. 1) discloses that dibutyl malate is synthesized while malic acid and n-butyl alcohol are taken as raw materials and solid superacid SO₄²⁻/TiO₂ are taken as a catalyst.

In addition, a Chinese patent document with publication number CN102369178A discloses a method for producing alpha-hydroxycarboxylic acid ester from alpha-hydroxycarboxylic acid amide and aliphatic alcohol, which is an industrially efficient production method in that the production cost is controlled while the conversion rate and selectivity are increased. Specifically, wherein the method for producing alpha-hydroxycarboxylic acid ester makes the alpha-hydroxycarboxylic acid amide and the aliphatic alcohol be subjected to gas-phase reaction while zirconium oxide is taken as a catalyst. It should be noted that the catalyst life is improved considerably by using the catalyst of the zirconium oxide containing specific elements. A Chinese patent document with publication number CN103687841A discloses a continuous method for producing alpha-hydroxycarboxylic acid esters, at least one alpha-hydroxycarboxylic acid amide, which is present in the liquid phase is reacted with an alcohol in the presence of a catalyst. The obtained alpha-hydroxycarboxylic acid esters are at least partially removed from the reaction mixture by means of the gas phase. A Chinese patent document with publication number CN112679347A discloses a method for preparing alpha-hydroxycarboxylic acid ester from hydrogen cyanide, the ammonia produced during the alcoholysis step of corresponding alpha-hydroxycarboxylic acid amide is recycled after a purification step for use in the hydrogen cyanide preparation process.

As mentioned above, there are problems in synthesis of alpha-hydroxycarboxylic acid ester as follows. When concentrated sulfuric acid, p-toluenesulfonic acid, sodium bisulfate and other cheap catalysts are used for an esterification reaction, there are problems such as equipment corrosion, large wastewater treatment capacity, and deep color of products. When solid acids and other catalysts are used for the esterification reaction, preparation of the catalysts is complex and products are difficult to regenerate, and the color of the products is deep. When the alpha-hydroxycarboxylic acid amide and the aliphatic alcohol are used as raw materials, the ammonia as a by-product of the reaction needs to be treated, and the process is complex.

### SUMMARY

In order to overcome shortcomings of preparation methods of alpha-hydroxycarboxylic acid ester in the prior art, the disclosure provides a convenient and environment-friendly synthesis method of alpha-hydroxycarboxylic acid ester.

Specifically, a synthesis method of alpha-hydroxycarboxylic acid ester includes following steps:
Step (1) a synthesis reaction, including: adding alpha-hydroxycarboxylic acid, alcohol, a catalyst and a water-carrying agent into a reaction device with an oil-water separator according to a mass ratio of the alpha-hydroxycarboxylic acid:the alcohol:the catalyst:the water-carrying agent being 100:(20-400):(0.1-10):(0-500), stirring under normal pressure, performing an esterification reaction at a temperature in a range of 100-160 Celsius degrees (° C.) for 3-10 hours, refluxing an oil layer in the oil-water separator, and distilling the water-carrying agent and unreacted alcohol when no water being produced in the oil-water separator, thereby obtaining a crude product; wherein the catalyst is one or more selected from a group consisting of boric acid, metaboric acid, pyroboric acid, and boric anhydride; and the water-carrying agent is one selected from a group consisting of cyclohexane, toluene, and benzene;
step (2), catalyst recovery, including: after the synthesis reaction, cooling the crude product to a room temperature, filtering out the catalyst to obtain a filtered product, and drying the catalyst for reuse;
step (3), neutralization and water washing, including: washing the filtered product obtained in the step (2) to neutral with water, or neutralizing the filtered product with an aqueous solution of sodium carbonate and then washing with water, to thereby obtain a neutral esterification product; and
step (4), refining, including: performing vacuum fractionation on the neutral esterification product obtained in the step (3) to obtain a refined product of the alpha-hydroxycarboxylic acid ester; wherein fractionated water-carrying agent and fractionated alcohol are used for a next reaction.

In an embodiment, the alpha-hydroxycarboxylic acid in the step (1) is one selected from a group consisting of lactic acid, mandelic acid, tartaric acid, citric acid, and malic acid.

In an embodiment, the alcohol in the step (1) is one selected from a group consisting of primary alcohol with an alkyl chain containing C1-C18 and secondary alcohol with an alkyl chain containing C1-C18.

In an embodiment, the catalyst in the step (1) is a composite catalyst composed of boric acid, and one of activated carbon and zeolite.

In an embodiment, the composite catalyst is prepared by placing the one of activated carbon and zeolite into a saturated aqueous solution of the boric acid for soaking for 2-3 hours and drying at a temperature of in a range of 100-150° C. for 3-5 hours.

In an embodiment, in the synthesis reaction of the step (1), when the oil-water separator separates water with 90-95% of a theoretical water yield of the esterification reaction, and the water-carrying agent and the unreacted alcohol are distilled by vacuum distillation; a vacuum degree of the vacuum distillation is −0.05 megapascals (MPa), and a temperature of the vacuum distillation is in a range of 80-130° C.

In an embodiment, after the crude product distilled the water-carrying agent and the unreacted alcohol is cooled to the room temperature, adding acetic anhydride into the cooled product, heating to a temperature in a range of 80-100° C., and performing an acetylation reaction for 3-8 hours; and distilling acetic acid and residual acetic anhydride by vacuum distillation after the acetylation reaction, wherein an amount of the acetic anhydride added into the cooled product is 1.5-3 times of an amount of alpha-hydroxyl groups of the crude product.

In an embodiment, one of sodium bisulfate and potassium bisulfate with mass being equal to 0.1-1% of mass of the alpha-hydroxycarboxylic acid ester is added into the acetylation reaction.

In an embodiment, the drying the catalyst in the step (2) specifically includes: drying the catalyst for 2-3 hours at a temperature in a range of 90-120° C.

In an embodiment, the refining in the step (4) specifically includes:
S1, exhausting air from a rectification tower to make a vacuum degree in the rectification tower be less than or equal to −0.10 MPa;
S2, delivering the neutral esterification product to a tower kettle of the rectification tower;
S3, heating to keep a temperature of the tower kettle at a temperature in a range of 100-130° C. and a temperature of a top of the rectification tower at a temperature in a range of 80-95° C., refluxing for 1-2 hours with a reflux ratio of 10-13:1 to collect the water-carrying agent and the unreacted alcohol;
S4, heating to keep the temperature of the tower kettle at a temperature in a range of 130-150° C. and the temperature of the top of the rectification tower at a temperature in a range of 95-105° C., refluxing with a reflux ratio of 15-20:1 to collect short chain alcohol ester (also referred to the alpha-hydroxycarboxylic acid ester with low boiling point, which is formed by the alpha-hydroxycarboxylic acid and short chain alcohols such as methanol, ethanol, propanol, butanol, and others.) of the alpha-hydroxycarboxylic acid;
S5, delivering the alpha-hydroxycarboxylic acid ester excluded short chain alcohol ester (also referred to the alpha-hydroxycarboxylic acid ester with high boiling point, which is formed by the alpha-hydroxycarboxylic acid and long chain alcohols such as octanol and palmitol.) of the tower kettle to a decolorization kettle, adding activated carbon with mass being equal to 2-3% of mass of the alpha-hydroxycarboxylic acid ester, keeping a temperature at 60-80° C., stirring for 1-1.5 hours, and separating out the activated carbon through a pressure filter to obtain the refined product of the alpha-hydroxycarboxylic acid ester.

The disclosure has following beneficial effects:
1. Different from the traditional synthesis methods of the alpha-hydroxycarboxylic acid ester, the disclosure uses one or more of corrosion-free, cheap and easily available boric acid, metaboric acid, pyroboric acid and boric anhydride as catalysts. After an esterification reaction, the neutral alpha-hydroxycarboxylic acid ester can be obtained by water washing or neutralization of the aqueous solution of sodium carbonate, which reduces the consumption of alkali during product neutralization and reduces discharge of wastewater.
2. The disclosure uses the boric acid catalyst and the like which has high catalytic activity and low consumption, and the esterification reaction can be performed smoothly when the catalysts are added with mass being equal to 0.1% of mass of the alpha-hydroxycarboxylic acid.
3. In the prior art, when concentrated sulfuric acid, p-toluenesulfonic acid, sodium bisulfate, solid acids and other catalysts are used, the hydroxycarboxylic acid is prone to dehydration and other side reactions under high-temperature conditions, and the product has a deep color. The disclosure adopts weak acidic boric acid catalysts, the product has light color, and the disclosure can produce colorless alpha-hydroxycarboxylic acid ester as a product, which improves product quality.
4. The boric acid catalyst and the like used in the method of the disclosure can catalyze alpha-hydroxycarboxylic acid to perform the esterification reaction and can also catalyze the alpha-hydroxycarboxylic acid to perform the acetylation reaction. Therefore, after the esterification reaction, the water-carrying agent and the unreacted alcohol can be removed by vacuum distillation, and the acetic anhydride can be directly added for the acetylation reaction, which reduces steps of the traditional preparation of acetylated alpha-hydroxycarboxylic acid ester and reduces the production cost.
5. The method of the disclosure is beneficial to promoting the generation of esterification products and the separation, recovery and utilization of catalysts by the composite catalysts composed of active carbon, zeolite and boric acid.

### DETAILED DESCRIPTION OF EMBODIMENTS

A variety of illustrated embodiments of the disclosure are described in detail. The detailed description should not be considered as a limitation of the disclosure, but should be understood as a more detailed description of some aspects, features and embodiments of the disclosure.

It should be understood that the terms described in the disclosure are only used to describe special embodiments, not to limit the disclosure. In addition, for a numerical range in the disclosure, it should be understood that each intermediate value between upper and lower limits of the range is also specifically disclosed. Each smaller range between intermediate values within any stated value or stated range and any other stated value or intermediate values within the range is also included in the disclosure. The upper and lower limits of the smaller range can be independently included or excluded from the range.

Unless otherwise stated, all technical and scientific terms used herein have the same meanings as those commonly understood by those skilled in the art described herein. Although the disclosure only describes preferred methods and materials, any methods and materials similar or equivalent to those described herein can be used in the implementation or testing of the disclosure. All documents mentioned in this specification are incorporated by reference to disclose and describe methods and/or materials related to the documents. In case of conflict with any incorporated literature, the contents of the specification shall prevail.

Without departing from the scope or spirit of the disclosure, it will be apparent to those skilled in the art that various improvements and changes can be made to the specific embodiments of the specification of the disclosure. Other embodiments obtained from the specification of the disclosure will be apparent to those skilled in the art. The specification and embodiments of the disclosure are only illustrative.

The words “include”, “comprise”, “have”, “contain”, etc. used in the specification are all open terms, which means including but not limited to.

Unless otherwise specified, the “part” described in the disclosure represents a part by mass.

The content of raw materials used in the embodiments of the disclosure can have a wide range. Components of the product can be determined qualitatively by gas chromatography-mass spectrometry (GC-MS), infrared spectroscopy and nuclear magnetic hydrogen spectroscopy. The relative content of volatile products is determined by referring to “GBT11, 538-2006 General Method for Determination of Essential Oil Capillary Column”. The refractive index and optical rotation of the product can be measured by a refractive index meter and an optical rotation meter.

### Embodiment 1

D-(−)-lactic acid, isooctanol, a catalyst and a water-carrying agent are added into a reaction device with an oil-water separator according to a mass ratio of the D-(−)-lactic acid:the isooctanol:the catalyst:the water-carrying agent being 40:78:0.6:40, stirred under normal pressure, and an esterification reaction is performed at a temperature in a range of 100-130° C. for 5 hours. An oil layer in the oil-water separator is refluxed, and the water-carrying agent and unreacted alcohol are distilled when no water is produced in the oil-water separator, thereby obtaining a crude product. The catalyst is boric acid, and the water-carrying agent is toluene. After the reaction, the crude product is cooled to a room temperature to obtain a cooled product, and the cooled product is washed to neutral with water to obtain a neutral esterification product. A refined product of lactate ester is obtained by performing vacuum fractionation on the neutral esterification product, and fractionated water-carrying agent and fractionated alcohol are used for a next reaction. Specific steps of the vacuum fractionation are as follows:
S1, exhausting air from a rectification tower to make a vacuum degree in the rectification tower be less than or equal to −0.10 megapascals (MPa);
S2, delivering the neutral esterification product (i.e., an esterification product of the D-(−)-lactic acid) to a tower kettle of the rectification tower;
S3, heating to keep a temperature of the tower kettle at a temperature in a range of 100-130° C. and a temperature of a top of the rectification tower at a temperature in a range of 80-95° C., refluxing for 2 hours with a reflux ratio of 10:1 to collect the toluene and the isooctanol; and
S4, heating to keep the temperature of the tower kettle at a temperature in a range of 130-150° C. and the temperature of the top of the rectification tower at a temperature in a range of 95-105° C., and refluxing with a reflux ratio of 15:1 to collect isooctyl lactate.

In this embodiment, properties of the isooctyl lactate are as follows: the purity is 99.5%, the yield is 98%, colorless, and the smell is aromatic; the measured optical rotation of the isooctyl lactate is +11.654° (20° C.), and the refractive index is 1.43280 (21.1° C.).

### Embodiment 2

L-(+)-lactic acid, n-butanol, a catalyst and a water-carrying agent are added into a reaction device with an oil-water separator according to a mass ratio of the L-(+)-lactic acid:the n-butanol:the catalyst:the water-carrying agent being 21:30:0.3:50, stirred under normal pressure, and an esterification reaction is performed at a temperature in a range of 100-130° C. for 5 hours; an oil layer in the oil-water separator is refluxed, and the water-carrying agent and unreacted alcohol are distilled when no water is produced in the oil-water separator, thereby obtaining a crude product; the catalyst is boric anhydride; the water-carrying agent is toluene; after the reaction, the crude product is cooled to a room temperature to obtain a cooled product; the cooled product is washed to neutral with water to obtain a neutral esterification product; a refined product of lactate ester is obtained by performing vacuum fractionation on the neutral esterification product; and fractionated water-carrying agent and fractionated alcohol are used for a next reaction. Specific steps of the vacuum fractionation are as follows:
S1, exhausting air from a rectification tower to make a vacuum degree in the rectification tower be less than or equal to −0.10 MPa;
S2, delivering the neutral esterification product (i.e., an esterification product of the L-(+)-lactic acid) to a tower kettle of the rectification tower;
S3, heating to keep a temperature of the tower kettle at a temperature in a range of 100-130° C. and a temperature of a top of the rectification tower at a temperature in a range of 80-95° C., refluxing for 1 hour with a reflux ratio of 10:1 to collect the toluene and the n-butanol; and
S4, heating to keep the temperature of the tower kettle at a temperature in a range of 130-150° C. and the temperature of the top of the rectification tower at a temperature in a range of 95-105° C., and refluxing with a reflux ratio of 16:1 to collect butyl lactate.

In this embodiment, properties of the butyl lactate are as follows: the purity is 99.5%, the yield is 98%, colorless, and the smell is aromatic; the measured optical rotation of the butyl lactate is −26.930° (20° C.), and the refractive index is 1.4485° (26.1° C.).

### Embodiment 3

R-(−)-mandelic acid, n-butanol, a catalyst and a water-carrying agent are added into a reaction device with an oil-water separator according to a mass ratio of the R-(−)-mandelic acid:the n-butanol:the catalyst:the water-carrying agent being 40:78:0.6:40, stirred under normal pressure, and an esterification reaction is performed at a temperature in a range of 100-130° C. for 5 hours; an oil layer in the oil-water separator is refluxed, and the water-carrying agent and unreacted alcohol are distilled when no water is produced in the oil-water separator, thereby obtaining a crude product; the catalyst is boric acid; the water-carrying agent is toluene; after the reaction, the crude product is cooled to a room temperature to obtain a cooled product; the cooled product is washed to neutral with water to obtain a neutral esterification product; a refined product of mandelate ester is obtained by performing vacuum fractionation on the neutral esterification product; and fractionated water-carrying agent and fractionated alcohol are used for a next reaction. Specific steps of the vacuum fractionation are as follows:
S1, exhausting air from a rectification tower to make a vacuum degree in the rectification tower be less than or equal to −0.10 MPa;
S2, delivering the neutral esterification product (i.e., an esterification product of the R-(−)-mandelic acid) to a tower kettle of the rectification tower;
S3, heating to keep a temperature of the tower kettle at a temperature in a range of 100-130° C. and a temperature of a top of the rectification tower at a temperature in a range of 80-95° C., refluxing for 1 hour with a reflux ratio of 10:1 to collect the toluene and the n-butanol; and
S4, heating to keep the temperature of the tower kettle at a temperature in a range of 130-150° C. and the temperature of the top of the rectification tower at a temperature in a range of 95-105° C., and refluxing with a reflux ratio of 18:1 to collect butyl mandelate.

In this embodiment, properties of the butyl mandelate are as follows: the purity is 99%, the yield is 90%, colorless, and the smell is aromatic; the measured optical rotation of the butyl mandelate is +77.894° (25° C.), and the refractive index is 1.4988° (26.1° C.).

### Embodiment 4

R-(−)-mandelic acid, n-butanol, a catalyst and a water-carrying agent are added into a reaction device with an oil-water separator according to a mass ratio of the R-(−)-mandelic acid:the n-butanol:the catalyst:the water-carrying agent being 26.8:44.4:1:50, stirred under normal pressure, and an esterification reaction is performed at a temperature in a range of 100-130° C. for 5 hours; an oil layer in the oil-water separator is refluxed, and the water-carrying agent and unreacted alcohol are distilled when no water is produced in the oil-water separator, thereby obtaining a crude product; the catalyst is a composite catalyst composed of boric acid and zeolite; the composite catalyst is prepared by placing the zeolite into a saturated aqueous solution of the boric acid for soaking for 3 hours and drying at a temperature of in a range of 140-150° C. for 3 hours; the water-carrying agent is toluene; after the reaction, the crude product is cooled to a room temperature to obtain a cooled product; the cooled product is washed to neutral with water to obtain a neutral esterification product; a refined product of mandelate ester is obtained by performing vacuum fractionation on the neutral esterification product; and fractionated water-carrying agent and fractionated alcohol are used for a next reaction. Specific steps of the vacuum fractionation are as follows:
S1, exhausting air from a rectification tower to make a vacuum degree in the rectification tower be less than or equal to −0.10 MPa;
S2, delivering the neutral esterification product (i.e., an esterification product of the R-(−)-mandelic acid) to a tower kettle of the rectification tower;
S3, heating to keep a temperature of the tower kettle at a temperature in a range of 100-130° C. and a temperature of a top of the rectification tower at a temperature in a range of 80-95° C., refluxing for 1 hour with a reflux ratio of 10:1 to collect the toluene and the n-butanol; and
S4, heating to keep the temperature of the tower kettle at a temperature in a range of 130-150° C. and the temperature of the top of the rectification tower at a temperature in a range of 95-105° C., and refluxing with a reflux ratio of 15:1 to collect butyl mandelate.

In this embodiment, properties of the butyl mandelate are as follows: the purity is 99.8%, the yield is 98%, colorless, and the smell is aromatic; the measured optical rotation of the butyl mandelate is +77.904° (25° C.), and the refractive index is 1.49880 (26.1° C.).

### Embodiment 5

L-(+)-tartaric acid, n-butanol, a catalyst and a water-carrying agent are added into a reaction device with an oil-water separator according to a mass ratio of the L-(+)-tartaric acid:the n-butanol:the catalyst:the water-carrying agent being 30:44.4:0.3:60, stirred under normal pressure, and an esterification reaction is performed at a temperature in a range of 100-130° C. for 5 hours; an oil layer in the oil-water separator is refluxed, and the water-carrying agent and unreacted alcohol are distilled when no water is produced in the oil-water separator, thereby obtaining a crude product; the catalyst is boric acid; the water-carrying agent is toluene; after the reaction, the crude product is cooled to a room temperature to obtain a cooled product; the cooled product is washed to neutral with water to obtain a neutral esterification product; a refined product of tartrate ester is obtained by performing vacuum fractionation on the neutral esterification product; and fractionated water-carrying agent and fractionated alcohol are used for a next reaction. Specific steps of the vacuum fractionation are as follows:
S1, exhausting air from a rectification tower to make a vacuum degree in the rectification tower be less than or equal to −0.10 MPa;
S2, delivering the neutral esterification product (i.e., an esterification product of the L-(+)-tartaric acid) to a tower kettle of the rectification tower;
S3, heating to keep a temperature of the tower kettle at a temperature in a range of 100-130° C. and a temperature of a top of the rectification tower at a temperature in a range of 80-95° C., refluxing for 1 hour with a reflux ratio of 10:1 to collect the toluene and the n-butanol; and
S4, heating to keep the temperature of the tower kettle at a temperature in a range of 130-150° C. and the temperature of the top of the rectification tower at a temperature in a range of 95-105° C., and refluxing with a reflux ratio of 18:1 to collect dibutyl tartrate.

In this embodiment, properties of the dibutyl tartrate are as follows: the purity is 99.0%, the yield is 90%, colorless, and the smell is aromatic; the measured optical rotation of the dibutyl tartrate is +6.976° (20° C.), and the refractive index is 1.44240 (25° C.).

### Embodiment 6

L-(+)-tartaric acid, n-butanol, a catalyst and a water-carrying agent are added into a reaction device with an oil-water separator according to a mass ratio of the L-(+)-tartaric acid:the n-butanol:the catalyst:the water-carrying agent being 30:44.4:0.3:60, stirred under normal pressure, and an esterification reaction is performed at a temperature in a range of 100-130° C. for 5 hours; an oil layer in the oil-water separator is refluxed, and the water-carrying agent and unreacted alcohol are distilled when no water is produced in the oil-water separator, thereby obtaining a crude product; the catalyst is a composite catalyst composed of boric acid and activated carbon; the composite catalyst is prepared by placing the activated carbon into a saturated aqueous solution of the boric acid for soaking for 2.5 hours and drying at a temperature of in a range of 110-130° C. for 3 hours; the water-carrying agent is toluene; after the reaction, the crude product is cooled to a room temperature to obtain a cooled product; the cooled product is washed to neutral with water to obtain a neutral esterification product; a refined product of tartrate ester is obtained by performing vacuum fractionation on the neutral esterification product; and fractionated water-carrying agent and fractionated alcohol are used for a next reaction. Specific steps of the vacuum fractionation are as follows:
S1, exhausting air from a rectification tower to make a vacuum degree in the rectification tower be less than or equal to −0.10 MPa;
S2, delivering the neutral esterification product (i.e., an esterification product of the L-(+)-tartaric acid) to a tower kettle of the rectification tower;
S3, heating to keep a temperature of the tower kettle at a temperature in a range of 100-130° C. and a temperature of a top of the rectification tower at a temperature in a range of 80-95° C., refluxing for 1 hour with a reflux ratio of 10:1 to collect the toluene and the n-butanol; and
S4, heating to keep the temperature of the tower kettle at a temperature in a range of 130-150° C. and the temperature of the top of the rectification tower at a temperature in a range of 95-105° C., and refluxingcollecting dibutyl tartrate.

In this embodiment, properties of the dibutyl tartrate are as follows: the purity is 99.8%, the yield is 96%, colorless, and the smell is aromatic; the measured optical rotation of the dibutyl tartrate is +6.976° (20° C.), and the refractive index is 1.44240 (25° C.).

### Embodiment 7

Citric acid, n-butanol, a catalyst and a water-carrying agent are added into a reaction device with an oil-water separator according to a mass ratio of the citric acid:the n-butanol:the catalyst:the water-carrying agent being 21:82:1:0, stirred under normal pressure, and an esterification reaction is performed at a temperature in a range of 100-130° C. for 6 hours; an oil layer in the oil-water separator is refluxed, and unreacted alcohol are distilled when no water is produced in the oil-water separator, thereby obtaining a crude product; the catalyst is boric acid; after the reaction, the crude product is cooled to a room temperature to obtain a cooled product; the cooled product is washed to neutral with water to obtain a neutral esterification product; a refined product of citrate ester is obtained by performing vacuum fractionation on the neutral esterification product; and fractionated alcohol are used for a next reaction. Specific steps of the vacuum fractionation are as follows:
S1, exhausting air from a rectification tower to make a vacuum degree in the rectification tower be less than or equal to −0.10 MPa;
S2, delivering the neutral esterification product (i.e., an esterification product of the citric acid) to a tower kettle of the rectification tower;
S3, heating to keep a temperature of the tower kettle at a temperature in a range of 100-130° C. and a temperature of a top of the rectification tower at a temperature in a range of 80-95° C., refluxing for 1 hour with a reflux ratio of 10:1 to collect the n-butanol; and
S4, heating to keep the temperature of the tower kettle at a temperature in a range of 130-150° C. and the temperature of the top of the rectification tower at a temperature in a range of 95-105° C., and refluxing with a reflux ratio of 20:1 to collect tributyl citrate.

In this embodiment, properties of the tributyl citrate are as follows: the purity is 98%, the yield is 96%, colorless; the measured refractive index of the tributyl citrate is 1.4433° (25° C.).

### Embodiment 8

Citric acid, n-butanol, a catalyst and a water-carrying agent are added into a reaction device with an oil-water separator according to a mass ratio of the citric acid:the n-butanol:the catalyst:the water-carrying agent being 21:66:0.2:0, stirred under normal pressure, and an esterification reaction is performed at a temperature in a range of 100-130° C. for 6 hours; an oil layer in the oil-water separator is refluxed, and unreacted alcohol are distilled when no water is produced in the oil-water separator, thereby obtaining a crude product; the catalyst is boric acid; after the reaction, the crude product is cooled to a room temperature to obtain a cooled product; the cooled product is washed to neutral with water to obtain a neutral esterification product; a refined product of citrate ester is obtained by performing vacuum fractionation on the neutral esterification product; and fractionated alcohol are used for a next reaction. Specific steps of the vacuum fractionation are as follows:
S1, exhausting air from a rectification tower to make a vacuum degree in the rectification tower be less than or equal to −0.10 MPa;
S2, delivering the neutral esterification product (i.e., an esterification product of the citric acid) to a tower kettle of the rectification tower;
S3, heating to keep a temperature of the tower kettle at a temperature in a range of 100-130° C. and a temperature of a top of the rectification tower at a temperature in a range of 80-95° C., refluxing for 1 hour with a reflux ratio of 10:1 to collect the n-butanol; and
S4, heating to keep the temperature of the tower kettle at a temperature in a range of 130-150° C. and the temperature of the top of the rectification tower at a temperature in a range of 95-105° C., and refluxing with a reflux ratio of 15:1 to collect tributyl citrate.

In this embodiment, the unreacted n-butanol acts as the water-carrying agent, and properties of the tributyl citrate are as follows: the purity is 98%, the yield is 90%, colorless; the measured refractive index of the tributyl citrate is 1.4433° (25° C.).

### Embodiment 9

Citric acid, n-butanol, a catalyst and a water-carrying agent are added into a reaction device with an oil-water separator according to a mass ratio of the citric acid:the n-butanol:the catalyst:the water-carrying agent being 42:74:0.04:0, stirred under normal pressure, and an esterification reaction is performed at a temperature in a range of 100-130° C. for 7 hours; an oil layer in the oil-water separator is refluxed, and unreacted alcohol are distilled when no water is produced in the oil-water separator, thereby obtaining a crude product; the catalyst is boric acid; after the reaction, the crude product is cooled to a room temperature to obtain a cooled product; the cooled product is washed to neutral with water to obtain a neutral esterification product; a refined product of citrate ester is obtained by performing vacuum fractionation on the neutral esterification product; and fractionated alcohol are used for a next reaction. Specific steps of the vacuum fractionation are as follows:
S1, exhausting air from a rectification tower to make a vacuum degree in the rectification tower be less than or equal to −0.10 MPa;
S2, delivering the neutral esterification product (i.e., an esterification product of the citric acid) to a tower kettle of the rectification tower;
S3, heating to keep a temperature of the tower kettle at a temperature in a range of 100-130° C. and a temperature of a top of the rectification tower at a temperature in a range of 80-95° C., refluxing for 1 hour with a reflux ratio of 10:1 to collect the n-butanol; and
S4, heating to keep the temperature of the tower kettle at a temperature in a range of 130-150° C. and the temperature of the top of the rectification tower at a temperature in a range of 95-105° C., and refluxing with a reflux ratio of 18:1 to collect tributyl citrate.

In this embodiment, the unreacted n-butanol acts as the water-carrying agent, and properties of the tributyl citrate are as follows: the purity is 99%, the yield is 85%, colorless; the measured refractive index of the tributyl citrate is 1.4432° (25° C.).

### Embodiment 10

Citric acid, n-butanol, a catalyst and a water-carrying agent are added into a reaction device with an oil-water separator according to a mass ratio of the citric acid:the n-butanol:the catalyst:the water-carrying agent being 21:82:0.1:0, stirred under normal pressure, and an esterification reaction is performed at a temperature in a range of 100-130° C. for 6 hours; an oil layer in the oil-water separator is refluxed, and unreacted alcohol are distilled when no water is produced in the oil-water separator, thereby obtaining a crude product; the catalyst is boric acid; cooling the crude product to a room temperature to obtain a cooled product, and then adding acetic anhydride into the cooled product; a mass ratio of the added acetic anhydride:the citric acid is 15:21; heating to a temperature in a range of 80-90° C. after adding the acetic anhydride, and performing an acetylation reaction for 3 hours to obtain an acetylated product; distilling acetic acid by vacuum distillation after the acetylation reaction under a vacuum degree of −0.1 MPa and a temperature of 80-90° C.; cooling the acetylated product to the room temperature, then the acetylated product is washed to neutral with water to obtain a neutral esterification product; a refined product of acetylated citrate ester is obtained by performing vacuum fractionation on the neutral esterification product; and fractionated alcohol are used for a next reaction. Specific steps of the vacuum fractionation are as follows:
S1, exhausting air from a rectification tower to make a vacuum degree in the rectification tower be less than or equal to −0.10 MPa;
S2, delivering the neutral esterification product (i.e., an esterification product of acetylated citric acid) to a tower kettle of the rectification tower;
S3, heating to keep a temperature of the tower kettle at a temperature in a range of 100-130° C. and a temperature of a top of the rectification tower at a temperature in a range of 80-95° C., refluxing for 1 hour with a reflux ratio of 10:1 to collect the n-butanol; and
S4, heating to keep the temperature of the tower kettle at a temperature in a range of 130-150° C. and the temperature of the top of the rectification tower at a temperature in a range of 95-105° C., and refluxing with a reflux ratio of 20:1 to collect acetylated tributyl citrate.

In this embodiment, the unreacted n-butanol acts as the water-carrying agent, and properties of the acetylated tributyl citrate are as follows: the purity is 99%, the yield is 98%, colorless; the measured refractive index of the tributyl citrate is 1.44200 (26° C.).

### Embodiment 11

Citric acid, n-octanol, a catalyst and a water-carrying agent are added into a reaction device with an oil-water separator according to a mass ratio of the citric acid:the n-octanol:the catalyst:the water-carrying agent being 42:130:0.2:0, stirred under normal pressure, and an esterification reaction is performed at a temperature in a range of 100-160° C. for 7 hours; an oil layer in the oil-water separator is refluxed, and unreacted alcohol are distilled when no water is produced in the oil-water separator, thereby obtaining a crude product; the catalyst is boric acid; after the reaction, the crude product is cooled to a room temperature to obtain a cooled product; the cooled product is washed to neutral with water to obtain a neutral esterification product; a refined product of citrate ester is obtained by performing vacuum fractionation on the neutral esterification product; and fractionated alcohol are used for a next reaction. Specific steps of the vacuum fractionation are as follows:
S1, exhausting air from a rectification tower to make a vacuum degree in the rectification tower be less than or equal to −0.10 MPa;
S2, delivering the neutral esterification product (i.e., an esterification product of the citric acid) to a tower kettle of the rectification tower;
S3, heating to keep a temperature of the tower kettle at a temperature in a range of 100-130° C. and a temperature of a top of the rectification tower at a temperature in a range of 80-95° C., refluxing for 2 hours with a reflux ratio of 13:1 to collect trioctyl citratethe and n-octanol; and
S4, delivering the trioctyl citrate of the tower kettle to a decolorization kettle, adding activated carbon with mass being equal to 2% mass of the trioctyl citrate, keeping a temperature at 60-80° C., stirring for 1.5 hours, and separating out the activated carbon through a pressure filter to obtain refined trioctyl citrate.

In this embodiment, the unreacted n-octanol acts as the water-carrying agent, and properties of the trioctyl citrate are as follows: the purity is 97%, the yield is 96%, colorless; the measured refractive index of the trioctyl citrate is 1.4533° (26° C.).

### Embodiment 12

Citric acid, isooctanol, a catalyst and toluene are added into a reaction device with an oil-water separator according to a mass ratio of the citric acid:the isooctanol:the catalyst:the toluene being 21:52:0.3:60, stirred under normal pressure, and an esterification reaction is performed at a temperature in a range of 100-130° C. for 8 hours; an oil layer in the oil-water separator is refluxed, and the toluene and unreacted alcohol are distilled when no water is produced in the oil-water separator, thereby obtaining a crude product; the catalyst is boric acid; after the reaction, the crude product is cooled to a room temperature to obtain a cooled product; the cooled product is washed to neutral with water to obtain a neutral esterification product; a refined product of citrate ester is obtained by performing vacuum fractionation on the neutral esterification product; and fractionated alcohol are used for a next reaction. Specific steps of the vacuum fractionation are as follows:
S1, exhausting air from a rectification tower to make a vacuum degree in the rectification tower be less than or equal to −0.10 MPa;
S2, delivering the neutral esterification product (i.e., an esterification product of the citric acid) to a tower kettle of the rectification tower;
S3, heating to keep a temperature of the tower kettle at a temperature in a range of 100-130° C. and a temperature of a top of the rectification tower at a temperature in a range of 80-95° C., refluxing for 2 hours with a reflux ratio of 13:1 to collect the isooctanol; and
S4, delivering the trioctyl citrate of the tower kettle to a decolorization kettle, adding activated carbon with mass being equal to 3% mass of the trioctyl citrate, keeping a temperature at 60-80° C., stirring for 1.5 hours, and separating out the activated carbon through a pressure filter to obtain refined trioctyl citrate.

In this embodiment, properties of the trioctyl citrate are as follows: the purity is 98%, the yield is 95%, colorless; the measured refractive index of the trioctyl citrate is 1.4531° (26° C.).

### Embodiment 13

Citric acid, n-octanol, a catalyst and a water-carrying agent are added into a reaction device with an oil-water separator according to a mass ratio of the citric acid:the n-octanol:the catalyst:the water-carrying agent being 42:120:0.3:0, stirred under normal pressure, and an esterification reaction is performed at a temperature in a range of 100-160° C. for 8 hours; an oil layer in the oil-water separator is refluxed, and unreacted alcohol are distilled when no water is produced in the oil-water separator, thereby obtaining a crude product; the catalyst is boric acid; cooling the crude product to a room temperature to obtain a cooled product, and then adding acetic anhydride into the cooled product; a mass ratio of the added acetic anhydride:the citric acid is 15:21; heating to a temperature in a range of 80-90° C. after adding the acetic anhydride, and performing an acetylation reaction for 3 hours to obtain an acetylated product; distilling acetic acid by vacuum distillation after the acetylation reaction under a vacuum degree of −0.1 MPa and a temperature of 80-90° C.; cooling the acetylated product to the room temperature, then the acetylated product is washed to neutral with water to obtain a neutral esterification product; a refined product of acetylated citrate ester is obtained by performing vacuum fractionation on the neutral esterification product; and fractionated alcohol are used for a next reaction. Specific steps of the vacuum fractionation are as follows:
S1, exhausting air from a rectification tower to make a vacuum degree in the rectification tower be less than or equal to −0.10 MPa;
S2, delivering the neutral esterification product (i.e., an esterification product of acetylated citric acid) to a tower kettle of the rectification tower;
S3, heating to keep a temperature of the tower kettle at a temperature in a range of 100-130° C. and a temperature of a top of the rectification tower at a temperature in a range of 80-95° C., refluxing for 2 hours with a reflux ratio of 13:1 to collect acetylated trioctyl citratethe and the n-octanol; and
S4, delivering the acetylated trioctyl citrate of the tower kettle to a decolorization kettle, adding activated carbon with mass being equal to 2.3% mass of the acetylated trioctyl citrate, keeping a temperature at 60-80° C., stirring for 1 hour, and separating out the activated carbon through a pressure filter to obtain refined acetylated trioctyl citrate.

In this embodiment, the unreacted n-octanol acts as the water-carrying agent, and properties of the acetylated trioctyl citrate are as follows: the purity is 96%, the yield is 90%, colorless; the measured refractive index of the acetylated trioctyl citrate is 1.4528° (26° C.).

### Embodiment 14

Malic acid, n-butanol, a catalyst and toluene are added into a reaction device with an oil-water separator according to a mass ratio of the malic acid:the n-butanol:the catalyst:the toluene being 26.8:44.4:0.3:50, stirred under normal pressure, and an esterification reaction is performed at a temperature in a range of 100-130° C. for 7 hours; an oil layer in the oil-water separator is refluxed, and the toluene and unreacted alcohol are distilled when no water is produced in the oil-water separator, thereby obtaining a crude product; the catalyst is boric acid; after the reaction, the crude product is cooled to a room temperature to obtain a cooled product; the cooled product is washed to neutral with water to obtain a neutral esterification product; a refined product of malate ester is obtained by performing vacuum fractionation on the neutral esterification product; and fractionated alcohol are used for a next reaction. Specific steps of the vacuum fractionation are as follows:
S1, exhausting air from a rectification tower to make a vacuum degree in the rectification tower be less than or equal to −0.10 MPa;
S2, delivering the neutral esterification product (i.e., an esterification product of the malic acid) to a tower kettle of the rectification tower;
S3, heating to keep a temperature of the tower kettle at a temperature in a range of 100-130° C. and a temperature of a top of the rectification tower at a temperature in a range of 80-95° C., refluxing for 1 hour with a reflux ratio of 10:1 to collect the n-butanol; and
S4, heating to keep the temperature of the tower kettle at a temperature in a range of 130-150° C. and the temperature of the top of the rectification tower at a temperature in a range of 95-105° C., and refluxing with a reflux ratio of 16:1 to collect dibutyl malate.

In this embodiment, properties of the dibutyl malate are as follows: the purity is 98%, the yield is 92%, colorless, the measured optical rotation of the dibutyl tartrate is −10.942° (20° C.), and the refractive index is 1.4388° (20° C.).

### Embodiment 15

Malic acid, n-butanol, a catalyst and toluene are added into a reaction device with an oil-water separator according to a mass ratio of the malic acid:the n-butanol:the catalyst:the toluene being 26.8:44.4:0.3:50, stirred under normal pressure, and an esterification reaction is performed at a temperature in a range of 100-130° C. for 7 hours; an oil layer in the oil-water separator is refluxed; when mass of water produced in the oil-water separator is equal to 90% theoretical mass, the toluene and unreacted alcohol are distilled with a vacuum degree of −0.05 MPa and a temperature in range of 80-130° C., thereby obtaining a crude product; the catalyst is boric acid; after the reaction, the crude product is cooled to a room temperature to obtain a cooled product; the cooled product is washed to neutral with water to obtain a neutral esterification product; a refined product of malate ester is obtained by performing vacuum fractionation on the neutral esterification product; and fractionated alcohol are used for a next reaction. Specific steps of the vacuum fractionation are as follows:
S1, exhausting air from a rectification tower to make a vacuum degree in the rectification tower be less than or equal to −0.10 MPa;
S2, delivering the neutral esterification product (i.e., an esterification product of the malic acid) to a tower kettle of the rectification tower;
S3, heating to keep a temperature of the tower kettle at a temperature in a range of 100-130° C. and a temperature of a top of the rectification tower at a temperature in a range of 80-95° C., refluxing for 1 hour with a reflux ratio of 10:1 to collect the n-butanol; and
S4, heating to keep the temperature of the tower kettle at a temperature in a range of 130-150° C. and the temperature of the top of the rectification tower at a temperature in a range of 95-105° C., and refluxing with a reflux ratio of 18:1 to collect dibutyl malate.

In this embodiment, properties of the dibutyl malate are as follows: the purity is 99%, the yield is 95%, colorless, and the smell is aromatic; the measured optical rotation of the dibutyl tartrate is −10.942° (20° C.), and the refractive index is 1.4388° (20° C.).

### Embodiment 16

R-(−)-mandelic acid, ethylene glycol, boric acid and toluene are added into a reaction device with an oil-water separator according to a mass ratio of the R-(−)-mandelic acid:the ethylene glycol:the boric acid:the toluene being 30.4:6.5:0.3:60, stirred under normal pressure, and an esterification reaction is performed at a temperature in a range of 100-130° C. for 8 hours; an oil layer in the oil-water separator is refluxed, and heating is stopped when no water is produced in the oil-water separator, thereby obtaining a crude product; after the reaction, the crude product is cooled to a room temperature to obtain a cooled product; the cooled product is washed to neutral with water to obtain a neutral esterification product; the toluene is distilled by vacuum fractionation with a vacuum degree of −0.05 MPa and a temperature in range of 80-110° C.; and ethylene glycol mandelate is obtained.

In this embodiment, properties of the ethylene glycol mandelate are as follows: the purity is 96%, the yield is 95%, colorless; the measured optical rotation of the dibutyl tartrate is +71.931° (25° C.), and the refractive index is 1.5381° (23° C.).

### Comparative Embodiment 1

Citric acid monohydrate, n-butanol are added into a reaction device with an oil-water separator according to a mass ratio of the citric acid monohydrate:the n-butanol being 21:66, stirred under normal pressure, and an esterification reaction is performed at a temperature in a range of 100-130° C. for 10 hours; an oil layer in the oil-water separator is refluxed; when no water is produced in the oil-water separator, mass of water separated by the oil-water separator is measured, it is found that mass of crystal water of citric acid raw material (i.e., the citric acid monohydrate) is equal to the separated water, and tributyl citrate is not detected by a chromatographic analysis.

The above described embodiments only describe preferred modes of the disclosure, not limit the scope of the disclosure. Without departing from the design spirit of the disclosure, all changes and improvements made by those ordinary skilled in the art to the technical solution of the disclosure should fall within the scope of protection determined in the claims of the disclosure.

What is claimed is:

1. A synthesis method of alpha-hydroxycarboxylic acid ester, comprising:
step (1), a synthesis reaction, comprising: adding alpha-hydroxycarboxylic acid, alcohol, a catalyst and a water-carrying agent into a reaction device with an oil-water separator according to a mass ratio of the alpha-hydroxycarboxylic acid:the alcohol:the catalyst:the water-carrying agent being 100:(20-400):(0.1-10):(0-500), stirring under normal pressure, performing an esterification reaction at a temperature in a range of 100-160 Celsius degrees (° C.) for 3-10 hours, refluxing an oil layer in the oil-water separator, and distilling the water-carrying agent and unreacted alcohol when no water being produced in the oil-water separator, thereby obtaining a crude product;
wherein the catalyst is one or more selected from a group consisting of boric acid, metaboric acid, pyroboric acid, and boric anhydride; and the water-carrying agent is one selected from a group consisting of cyclohexane, toluene, and benzene;
step (2), catalyst recovery, comprising: after the synthesis reaction, cooling the crude product to a room temperature, filtering out the catalyst to obtain a filtered product, and drying the catalyst for reuse;
step (3), neutralization and water washing, comprising: washing the filtered product obtained in the step (2) to neutral with water, or neutralizing the filtered product with an aqueous solution of sodium carbonate and then washing with water, to thereby obtain a neutral esterification product; and
step (4), refining, comprising: performing vacuum fractionation on the neutral esterification product obtained in the step (3) to obtain a refined product of the alpha-hydroxycarboxylic acid ester; wherein fractionated water-carrying agent and fractionated alcohol are used for a next reaction.

2. The synthesis method of alpha-hydroxycarboxylic acid ester according to claim 1, wherein the alpha-hydroxycarboxylic acid in the step (1) is one selected from a group consisting of lactic acid, mandelic acid, tartaric acid, citric acid, and malic acid.

3. The synthesis method of alpha-hydroxycarboxylic acid ester according to claim 1, wherein the alcohol in the step (1) is one selected from a group consisting of primary alcohol with an alkyl chain containing C1-C18 and secondary alcohol with an alkyl chain containing C1-C18.

4. The synthesis method of alpha-hydroxycarboxylic acid ester according to claim 1, the catalyst in the step (1) is a composite catalyst composed of boric acid, and one of activated carbon and zeolite.

5. The synthesis method of alpha-hydroxycarboxylic acid ester according to claim 4, wherein the composite catalyst is prepared by placing the one of activated carbon and zeolite into a saturated aqueous solution of the boric acid for soaking for 2-3 hours and drying at a temperature of in a range of 100-150° C. for 3-5 hours.

6. The synthesis method of alpha-hydroxycarboxylic acid ester according to claim 1, wherein in the synthesis reaction of the step (1), when the oil-water separator separates water with 90-95% of a theoretical water yield of the esterification reaction, and the water-carrying agent and the unreacted alcohol are distilled by vacuum distillation; a vacuum degree of the vacuum distillation is −0.05 megapascals (MPa), and a temperature of the vacuum distillation is in a range of 80-130° C.

7. The synthesis method of alpha-hydroxycarboxylic acid ester according to claim 6, further comprising:
after the crude product distilled the water-carrying agent and the unreacted alcohol is cooled to the room temperature, adding acetic anhydride into the cooled product, heating to a temperature in a range of 80-100° C., and performing an acetylation reaction for 3-8 hours; and
distilling acetic acid and residual acetic anhydride by vacuum distillation after the acetylation reaction, wherein an amount of the acetic anhydride added into the cooled product is 1.5-3 times of an amount of alpha-hydroxyl groups of the crude product.

8. The synthesis method of alpha-hydroxycarboxylic acid ester according to claim 7, wherein one of sodium bisulfate and potassium bisulfate with mass being equal to 0.1-1% of mass of the alpha-hydroxycarboxylic acid ester is added into the acetylation reaction.

9. The synthesis method of alpha-hydroxycarboxylic acid ester according to claim 1, wherein the drying the catalyst in the step (2) specifically comprises: drying the catalyst for 2-3 hours at a temperature in a range of 90-120° C.

10. The synthesis method of alpha-hydroxycarboxylic acid ester according to claim 1, wherein the refining in the step (4) specifically comprises:
S1, exhausting air from a rectification tower to make a vacuum degree in the rectification tower be less than or equal to −0.10 MPa;
S2, delivering the neutral esterification product to a tower kettle of the rectification tower;
S3, heating to keep a temperature of the tower kettle at a temperature in a range of 100-130° C. and a temperature of a top of the rectification tower at a temperature in a range of 80-95° C., refluxing for 1-2 hours with a reflux ratio in a range of 10-13:1 to collect the water-carrying agent and the unreacted alcohol;
S4, heating to keep the temperature of the tower kettle at a temperature in a range of 130-150° C. and the temperature of the top of the rectification tower at a temperature in a range of 95-105° C., refluxing with a reflux ratio of 15-20:1 to collect short chain alcohol ester of the alpha-hydroxycarboxylic acid;
S5, delivering the alpha-hydroxycarboxylic acid ester excluded short chain alcohol ester of the tower kettle to a decolorization kettle, adding activated carbon with mass being equal to 2-3% of mass of the alpha-hydroxycarboxylic acid ester, keeping a temperature at 60-80° C., stirring for 1-1.5 hours, and separating out the activated carbon through a pressure filter to obtain the refined product of the alpha-hydroxycarboxylic acid ester.
